(19) United States
(12) Patent Application Publication
(10) Pub. No.: US 2024/0075230 A1
(43) Pub. Date: Mar. 7, 2024

(54) INTUBATION DEVICES AND SYSTEMS

(71) Applicant: Flexicare (Group) Limited, Mid Glamorgan (GB)

(72) Inventors: Ben Skirrow, Mid Glamorgan (GB); Tudor Thomas, Mid Glamorgan (GB)

(21) Appl. No.: 18/200,741

(22) Filed: May 23, 2023

(30) Foreign Application Priority Data
Sep. 2, 2022 (GB) 2212813.6

(51) Int. Cl. A61M 16/04

(52) U.S. Cl. CPC A61M 16/0488

(57) ABSTRACT

Provided herein is an endotracheal tube, and an intubation system comprising said endotracheal tube and a stylet for guiding an endotracheal tube during intubation. The endotracheal tube has a body comprising a flexible hollow tube with a distal end for insertion into a patient's trachea during intubation, and an opposite proximal end. The endotracheal tube body comprises a polymeric material comprising a helically wound, meshed or braided reinforcement structure embedded therein, the helically wound, meshed or braided reinforcement structure being formed from one or more filaments. One or more properties of the reinforcement structure vary along the length of the endotracheal tube such that the bending flexibility and/or the mechanical strength of the endotracheal tube varies along the length of the endotracheal tube.

### RELATED APPLICATION

This application claims priority to United Kingdom Patent Application GB 2212813.6 entitled Intubation Devices and Systems filed Sep. 2, 2022, the entire disclosure of which is expressly incorporated herein by reference.

### FIELD OF THE INVENTION

The present invention relates to intubation devices. In particular, it relates to endotracheal tubes, and intubation systems incorporating such endotracheal tubes.

### BACKGROUND

This United States Patent Application is filed on even date with copending U.S. patent application Ser. No. 18/200,730 entitled Intubation Devices and Systems which claims priority to UK Patent Application GB221807.8 filed Sep. 2,2022, the entire disclosure of which is expressly incorporated herein by reference.

During tracheal intubation, an endotracheal tube (ET Tube) must be inserted into a patient's airway. In a typical intubation process, a clinician standing above/behind the head of the supine patient will use a laryngoscope to move the tongue and epiglottis out of the way before insertion of an ET tube through the patient's vocal cords into the trachea. Once the ET tube is correctly positioned in the trachea, commonly a cuff on the ET tube will then be inflated to hold the tube in position for ventilation of the patient. It is necessary to ensure accurate placement of the ET tube in the trachea, and to avoid incorrect placement of the ET tube into e.g. the oesophagus rather than the trachea. The patient is commonly anaesthetised and apnoeic, so the intubation procedure has to be completed rapidly, and it is advantageous to quickly and accurately confirm correct placement of the ET tube so ventilation can begin.

Location of the ET tube can be determined using a number of methods, including visualisation, capnography, and X-ray location of the tube, alongside physical examination methods such as auscultation of the chest and epigastrium, and visualisation of thoracic movement. However, many of these methods of determining ET tube placement are not sufficiently reliable to be used as sole techniques to correctly determine ET tube location. Visualisation of the ET tube passing through the vocal cords into the trachea offers the most reliable method of quickly and accurately determining correct placement of the ET tube.

In some cases, the clinician performing the intubation may be able to directly visualise placement of the ET tube, however this is not always possible, depending on the particular anatomy of the patient. For example, difficulties may be encountered where the patient has restricted neck flexibility, or where the patient is obese. For such patients, video laryngoscopes are well-known as one option for facilitation of intubation. However, video laryngoscopes suffer from a number of problems. As video apparatus is typically provided at an intermediate location along the laryngoscope blade, the distal end of the blade can partially obstruct the field of view. Additionally, the ET tube itself may obstruct the view of the vocal cords and trachea as it advances past the end of the laryngoscope. Video laryngoscopes can also be relatively high in cost, which limits their application as single use devices.

The act of placing the ET tube offers its own difficulties, even where it can be visualised clearly. Typically, an ET tube is made from semi-rigid polymer, and has a gentle curve to align with the airways of the patient. However, patient anatomy may require that the ET tube has a specific shape, for example, a sharper bend at the distal end, to aid insertion through the vocal cords into the trachea. Because the material of the ET tube is generally flexible and typically does not retain a shape when bent, a stylet may be used in combination with the ET tube. A stylet is a device that can be inserted into an ET tube to alter the shape of an ET tube to facilitate intubation. Stylets may also provide additional rigidity to the ET tube to aid navigation of the ET tube into the desired location. Some known stylets can be inserted into the ET tube and shaped by the clinician so the form of the ET tube is retained before and during insertion of the combined devices into the patient's airway. However, with these stylets, the shape of the stylet must be set before intubation occurs and can lead to undesirable delays during the intubation procedure where the shape is not quite appropriate for the patient's anatomy.

To overcome one or more of the above problems, stylets which offer an amount of adjustability during the intubation procedure have been provided previously. For example GB2563567B discloses a stylet comprises a body having a pivotable tip located at a distal end of the body. This type of stylet is referred to as a ‘pivot-end’ type stylet or an articulated stylet. The terms may be used interchangeably. Such stylets may also provide imaging capabilities.

However, conventional ET tubes may not be particularly suitable for use with this type of stylet: they may not have sufficient bending flexibility. Alternatively, where they do have sufficient bending flexibility, they may be prone to risk of collapse on inflation of the ET tube cuff, which can pose risks to patient safety. For example, whilst GB2563567B also discloses an ET tube which is particularly suitable for use with this type of stylet, where the ET tube has a bending portion or local flexure at the distal end of the tube, it has been found that such bending/flexure arrangements may be prone to risk of collapse on inflation of the ET tube cuff, which can pose risks to patient safety.

A further problem that arises in use of conventional ET tubes with stylets having imaging capabilities, is that it can be difficult to ensure that the field of view of the stylet is not obscured during use e.g. by a tip portion of the ET tube. This problem is exacerbated for pivot-end/articulated stylets: it can be especially difficult to prevent field of view obscuration whilst retaining the full range of motion for the stylet. This can also pose risks to patient safety, e.g. where a clinician can not clearly visualise the patient's airway during an intubation. It would be desirable to provide an ET tube which both offers suitable bending flexibility for use with a wide variety of stylets, including ‘pivot-end’-type/articulated stylets, and which furthermore offers improvements in respect of patient safety during use of the ET tube.

The present invention has been devised in light of the above considerations.

### SUMMARY OF THE INVENTION

The present inventors have realised that providing an endotracheal tube (also referred to herein as an ‘ET’ tube) having a meshed or braided reinforcement structure embedded therein may help to overcome some or all of the above problems. Furthermore, they have realised that it would be advantageous to provide an endotracheal tube where the bending response of the tube and/or the mechanical strength of the ET tube is customised along its length.

Accordingly, in a first aspect, the present invention provides an endotracheal tube having a body comprising a flexible hollow tube with a distal end for insertion into a patient's trachea during intubation, and an opposite proximal end, wherein: the endotracheal tube body comprises a polymeric material comprising a helically wound, meshed or braided reinforcement structure embedded therein, the helically wound, meshed or braided reinforcement structure being formed from one or more filaments, and wherein one or more properties of the reinforcement structure vary along the length of the endotracheal tube such that the bending flexibility and/or the mechanical strength of the endotracheal tube varies along the length of the endotracheal tube.

In the present disclosure, various features of the present invention are described in relation to directions defined relative to the general shape of the endotracheal tube. In this sense, the endotracheal tube can be considered to have a longitudinal direction (extending along a longitudinal length of the tube), a circumferential direction (extending about a circumference of the tube), and a radial direction (extending outwardly from the centre of the tube).

By providing an endotracheal tube in which the tube body comprises a helically wound, meshed or braided reinforcement structure embedded therein, wherein the reinforcement structure is configured such that the bending flexibility and/or the mechanical strength of the endotracheal tube varies along the length of the endotracheal tube, it is possible to provide an endotracheal tube which provides both suitable bending flexibility and/or the mechanical strength for use with a wide variety or stylets or other intubation aids, whilst also providing suitable resistance to collapse of the endotracheal tube in different regions of the tube.

The bending flexibility and the mechanical strength of the tube along its length can be characterized by any suitable methods. In one example, the bending flexibility may suitably be characterised using a 3-point bend test, for example as set out in ASTM D790 or ISO 178.

Where convenient, the bending flexibility may be defined by the peak torque recorded on a torque gauge (e.g. H10S digital torque meter) on actuation of a control member of an articulated stylet (e.g. of the type described in GB2563567B) disposed within a central lumen of the ET tube during flexion/retroflexion of the ET tube by control of the articulated stylet.

As noted above, the bending flexibility and/or the mechanical strength of the endotracheal tube can be varied by changing one or more properties of the reinforcement structure along the length of the endotracheal tube. The one or more properties that can be varied may include: the material of the filament(s) forming the reinforcement structure; the cross-sectional shape or area of the filament(s) forming the reinforcement structure; and/or the relative volume proportion of the filament(s) forming the reinforcement structure within the endotracheal tube (e.g. length/volume of filament per length/volume of endotracheal tube).

The one or more filaments forming the helically wound, meshed or braided reinforcement structure may be formed from any suitable material. In some embodiments the filaments comprise a metallic material. In other embodiments, the filaments comprise a polymeric material. In yet other embodiments, the filaments may comprise an aramid or para-aramid material, or a carbon fibre material. Preferably the filaments are formed from metal. For example they may be formed from a material selected from the group consisting of: stainless steel, Kevlar, HDPE, and carbon fibre. In preferred embodiments, the filaments may be formed from stainless steel. As noted above, the material of the filaments may in some embodiments vary along the length of the endotracheal tube—either the material may vary along the length of the or each filament forming part of the reinforcement structure, or a plurality of filaments may be provided, wherein a first set of filaments are made from a first material, and a second set of filaments are formed from a second material (different to the first material), and wherein the first and second set of filaments are arranged such that the such the bending flexibility and/or the mechanical strength of the endotracheal tube varies along the length of the endotracheal tube.

The filaments forming the helically wound, meshed or braided reinforcement structure may have a substantially circular cross-sectional shape (in a cross-section taken perpendicular to the direction of extension of the filaments). Alternatively they may have a flattened cross-sectional shape. For example, the filaments may have an oval, oblong, or rectangular cross-sectional shape in a cross-section taken perpendicular to the direction of extension of the filaments. The use of an elliptical or rectangular cross section may be advantageous where it is desired to provide a higher axial stiffness and length stability in comparison to the flexural stiffness. As noted above, the cross-sectional shape or area of the filaments may in some embodiments vary along the length of the endotracheal tube—either the cross-sectional shape or area may vary along the or each filament forming part of the reinforcement structure, or a plurality of filaments may be provided, wherein a first set of filaments have a first cross-sectional shape or area, and a second set of filaments have a second cross-sectional shape or area (different to the first material), and wherein the first and second set of filaments are arranged such that the such the bending flexibility and/or the mechanical strength of the endotracheal tube varies along the length of the endotracheal tube.

The filaments forming the helically wound, meshed or braided reinforcement structure may have largest dimension in a direction perpendicular to the longitudinal extension of the filaments in a range of from 0.1 to 1 mm, preferably 0.1 to 0.5 mm, e.g. 0.1 mm or more, 0.2 mm or more, 0.3 mm or more, 0.4 mm or more or 0.5 mm or more, or 1 mm or less, 0.9 mm or less, 0.8 mm or less, 0.7 mm or less, 0.6 mm or less, or 0.5 mm or less. For example, where the filaments forming the helically wound, meshed or braided reinforcement structure have a substantially circular cross section, they may have a diameter in a range of from 0.1 to 0.5 mm, or any of the other ranges set out above. Where the filaments forming the helically wound, meshed or braided reinforcement structure have a substantially rectangular cross section, they may be 0.2 mm thick in the radial direction and 0.4 mm wide in the circumferential direction of the ET tube. It has been found that providing filaments of such dimensions can provide for a suitable balance between bending stiffness and resistance to collapse of the ET tube.

In preferred arrangements, the reinforcement structure is provided by one or more helically wound filaments. The one or more helically wound filaments may be arranged in parallel and/or in series along the length of the ET tube. In one suitable arrangement, the reinforcement structure may be provided by a single filament which is helically wound along at least part of the endotracheal tube body. A spiral/helical wound filament can provide suitable flexibility for the endotracheal tube body whilst still preventing collapse and/or kinking of the endotracheal tube during use. Furthermore, a reinforcement structure comprising non-overlapping reinforcing filaments can provide for low profile reinforcement, thereby allowing the walls of the endotracheal tube to have a relatively low cumulative thickness as compared with the use of other reinforcement structures (e.g. meshed structures with overlapping filaments). Providing a low wall thickness allows a larger central lumen diameter for a given outer diameter of endotracheal tube, allowing for suitably low flow resistance during use, thereby minimising burden on the patient of the effort of exhalation.

Where the reinforcement structure comprises one or more helically wound filaments, the pitch of the helical winding of each filament may be in a range of from 0.75 mm to 5 mm, preferably 0.75 mm to 3 mm, preferably about 1 mm to 2 mm. For example, the pitch may be 0.75 mm or more, 0.8 mm or more, 0.9 mm ore more 1 mm or more, 1.5 mm or more, 2 mm or more, or 2.5 mm or more. The pitch may be 3 mm or less, 2.5 mm or less, 2 mm or less, 1.5 mm or less, or 1 mm or less. In one suitable arrangement, the pitch may be about 1.3 mm. The pitch is measured as the length of one complete helix turn in a longitudinal direction along the ET tube. The pitch may be substantially constant along the length of the endotracheal tube. Alternatively, the pitch may vary along the length of the endotracheal tube. Variation in pitch of the helically wound filament(s) along the length of the tube is one way of varying the length/volume of filament per length/volume of endotracheal tube to thereby achieve a desired bending response for the endotracheal tube: providing a smaller pitch equates to providing a larger length of filament per length of endotracheal tube. Providing a larger pitch equates to providing a shorter length of filament per length of endotracheal tube. Where the pitch varies along the length of the endotracheal tube, it may vary in a continuous/gradual manner, or may alternatively vary in a step-wise manner. Varying the pitch continuously may advantageously allow for a smooth change in bending response/mechanical strength along the tube. Varying the pitch in a step-wise manner may provide a simpler manufacturing process.

In one preferred arrangement, two or more distinct helically wound filaments (e.g. in the form of helical springs) may be sequentially arranged along the length of the endotracheal tube body, thereby providing the reinforcement structure. A property of a first helically wound filament may differ from a property of a second helically wound filament to thereby provide an arrangement in which the bending flexibility of the endotracheal tube varies along the length of the endotracheal tube. In a particularly preferred arrangement, a first helically wound filament is arranged distally of a second helically wound filament along the length of the endotracheal tube, and the pitch of the first helically wound filament is greater than the pitch of the second helically wound filament. Such arrangements may ensure resistance to crushing and kinking of the endotracheal tube in a proximal region of the tube (e.g. a region which in use, extends from the upper airway to outside the patient's body), which allowing a more flexible cross-section in the distal portion of the tube to ensure optimal flexibility of the tube in this region whilst providing adequate resistance to collapse. Similar advantages may also be achieved by provided an arrangement in which the first helically wound filament comprises a thinner filament cross-sectional area than the second helically wound filament, and/or a different cross-sectional shape than the second helically wound filament. In some arrangements, both the pitch and the cross-sectional area and/or shape of the relevant filaments may be varied along the length of the endotracheal tube.

The use of sequentially arranged and separate helically wound filaments (e.g. helical springs) having different properties may be advantageous over arrangements in which one or more properties of a single filament are varied along its length. In particular, such an arrangement may facilitate ease of manufacture of the reinforcement structure whilst retaining the ability to vary mechanical properties of the ET tube along its length.

Where the reinforcement structure is provided as a meshed or braided structure, this may alternatively be referred to as a reinforcement network. The precise form of the mesh or braid is nor particularly limited, and many suitable arrangements can be contemplated. In one arrangement, the filaments forming the helically wound, meshed or braided reinforcement structure may comprise two or more set of filaments, wherein substantially all filaments in a given set of filaments extend longitudinally in parallel to one another. One or more properties of the sets of filaments may differ. For example, the direction of extension, size or spacing of filaments in the first set of filaments may differ from that of the second set of filaments. For example, the helically wound, meshed or braided reinforcement structure may comprise a first set of filaments which extend longitudinally in a first direction. The helically wound, meshed or braided reinforcement structure may comprise a second set of filaments which extend longitudinally in a second direction. In such an arrangement, the second direction is preferably different to the first direction.

The first direction may be arranged at an angle α of from 0° to 90° with respect to the longitudinal axis of the endotracheal tube. An angle of 0° with respect to the longitudinal axis of the endotracheal tube means that the first direction is parallel to the longitudinal axis of the endotracheal tube. An angle of 90° with respect to the longitudinal axis of the endotracheal tube means that the first direction is perpendicular to the longitudinal axis of the endotracheal tube.

The second direction may be arranged at an angle β of from 0° to 90° with respect to the longitudinal axis of the endotracheal tube. An angle of 0° with respect to the longitudinal axis of the endotracheal tube means that the first direction is parallel to the longitudinal axis of the endotracheal tube. An angle of 90° with respect to the longitudinal axis of the endotracheal tube means that the first direction is perpendicular to the longitudinal axis of the endotracheal tube.

As noted above, preferably the first direction is different to the second direction—that, is preferably, α is not equal to β.

The helically wound, meshed or braided reinforcement structure may extend along substantially an entire length of the endotracheal tube. Alternatively, the helically wound, meshed or braided reinforcement structure may extend along only part of the length of the endotracheal tube. For example, the helically wound, meshed or braided reinforcement structure may extend along at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the length of the endotracheal tube. The part of the tube along which the reinforcement structure extends may be a proximal part of the tube, e.g. a part of the tube extending from a proximal end of the tube towards the distal tip end. In some arrangements, the reinforcement network may be provided along the ET tube body up to but not including the distal tip portion of the tube. This can allow the distal tip portion to be provided with features such as murphy eyes, which are side apertures that enable gas flow even if the tube tip is mispositioned and blocked and act as a back-up gas flow path. Furthermore, arrangements in which the reinforcement network does not extend all the way to the distal tip of the endotracheal tube may also be advantageous from a patient safety perspective: providing a reinforcement-structure-free distal tip end can reduce the risk of part of the reinforcement structure (e.g. a wire) puncturing the material of the endotracheal tube at the distal tip end which could cause trauma to a patient.

Where the ET tube body comprises a main body portion and a distinct distal tip portion which are separate components, the helically wound, meshed or braided reinforcement structure may be provided in one or both of the main body and the distal tip portions, as noted above. However, in preferred arrangements, the helically wound, meshed or braided reinforcement structure is provided only in the main body portion of the endotracheal tube. Such an arrangement can allow for improved bending flexibility of the distal tip portion of the tube, and furthermore may allow for ease of mass manufacture of the ET tube. The same benefits relating to improved patient safety noted above are also provided by such arrangements.

The present inventors have also found that provision of a reinforcement structure in combination with one or more internal projections may provide an even further improved arrangement for an endotracheal tube.

Accordingly, in a second aspect, the present invention provides an endotracheal tube having a body comprising a flexible hollow tube with a distal end for insertion into a patient's trachea during intubation, and an opposite proximal end, wherein: the endotracheal tube body comprises a polymeric material comprising a helically wound, meshed or braided reinforcement structure embedded therein, the helically wound, meshed or braided reinforcement structure being formed from one or more filaments; and
wherein the endotracheal tube further comprises one or more internal projections, said internal projections projecting radially inwardly from an internal wall surface of a distal tip portion of the endotracheal tube.

The phrase ‘projecting radially inwardly’ will be understood as defining that the projections extend inwardly from an internal wall surface in a radial direction, towards a central longitudinal axis of the ET tube.

The provision of one or more internal projections may both assist in ensuring correct alignment of stylets or other intubation aids within the endotracheal tube during use, whilst further reducing the risk of total collapse of the endotracheal tube. Endotracheal tubes comprising a combination of a helically wound, meshed or braided reinforcement structure, and one or more internal projections are therefore found to offer particular advantages in respect of improved patient safety during use of the endotracheal tube, as compared with known arrangements.

In some embodiments, the internal projections may be tapered in at least one direction. The term “tapered” is used herein to define that a dimension of the projections diminishes or reduces along the specified direction. The projections may be tapered along their entire length in the specified direction. Alternatively, the projections may be tapered along a part of their length in the specified direction. That is, the projections may comprise a tapered portion. The tapering nature of the projections may result in the projections comprising a ramped cross-sectional profile. That is, alternatively or additional to describing the projections as being ‘tapered’, the projections may be defined as comprising one or more ramp surfaces.

However, the use of tapered projections is not considered to be essential, and other forms of projection may be used. For example, in some arrangements, the projections may be generally hemispherical, or hemicylindrical. The use of rounded shapes like this may be advantageous as it can reduce the risk of a stylet, or any other components intended to pass through the central lumen of the ET tube getting stuck or caught on the projections. This may be advantageous in particular for arrangements in which it is desirable to allow the stylet or other component to pass through the lumen of the ET tube. In other arrangements, the projections may be e.g. substantially cuboid in shape.

The provision of one or more such internal projections may both assist in ensuring correct alignment of video stylets or other intubation aids within the endotracheal tube during use, whilst further reducing the risk of total collapse of the endotracheal tube, and additionally allowing the use of softer grades of material which can reduce or mitigate risk of tissue trauma during use of the endotracheal tube due to additional flexural stiffness provided by the projections. The use of projections having a tapered shape has been found to be particularly advantageous as it can provide a ramp surface allowing for even further improved alignment of video stylets or other intubation aids within the endotracheal tube during use.

The specific size and/or shape of the projections may be selected as appropriate for the specific intended application of the ET tube. In some preferred arrangements, the projections may be elongate ribs. An elongate rib is defined herein as a member in which the length of said member is substantially larger than at least one other dimension of the member, e.g. is substantially larger than the height and/or the width of the member. In some arrangements, the length of the member may be at least twice the height and/or the width of the member, or at least three, four, or five times the height and/or width of the member. Further details regarding preferred geometry of the projections are set out below.

Suitably, the projections may comprise elongate ribs having an extension direction which is substantially parallel to a longitudinal axis of the endotracheal tube. Such arrangements may increase the flexural stiffness of the endotracheal tube in comparison to arrangements where projections are aligned to extend e.g. in a circumferential direction. This may be advantageous as it may allow for softer grade materials to be used for the ET tube, whilst retaining a suitable overall flexural stiffness.

Preferably, the internal projections are tapered along their extension direction. That is, the height of the internal projections (measured in a radial direction of the ET tube) may vary along the extension direction in a tapered manner. In preferred arrangements, the internal projections may be tapered such that a proximal portion of the projections (i.e. a portion closer to a proximal end of the ET tube) has a lower height than a distal portion of the projections (i.e. a portion closer to a distal end of the ET tube).

In some arrangements, the projections may comprise a first portion which is tapered in a first direction, and a second portion which is tapered in a second direction, different to the first direction. The first and second direction may be opposition directions. Where the projections taper in first and second directions, the gradient of tapering may differ in the first and second directions.

In one particularly preferred arrangement, a proximal portion of the projections is tapered such that the height of the proximal portion decreases towards the proximal end of the ET tube, and a distal portion of the projections is tapered such that the height of the distal portion decreases towards the distal end of the ET tube. In such arrangements, the projections may have a maximum height at an intermediate point along their length, i.e. a maximum height at a point intermediate the proximal and distal portions of the projections. The distal portion of the projections may have a steeper gradient of taper than the proximal portion of the projections.

The shape of the projections in a cross-section perpendicular to the longitudinal axis of the ET tube is not particularly limited. In some arrangements the projections may have a substantially square, rectangular, triangular, semicircular, or elliptical cross-sectional profile in a cross-section perpendicular to the longitudinal axis of the ET tube.

The size of the projections may be selected as appropriate for the intended use of the ET tube. In some arrangements, the size of the projections is selected to reduce or minimize the impact of the projections on the flow of respiratory gases during use of the ET tube, or to otherwise prevent obstruction of the tube.

Alternatively or additionally, the size of the projections may be selected to prevent passage of articles of a predetermined dimension from passing through the ET tube past the projections. That is, the projections may be configured to restrict a central lumen of the ET tube to have a predetermined maximum dimension in at least one direction. The predetermined maximum dimension may be a dimension in a range of from 4 mm to 7 mm. More preferably the predetermined maximum dimension of the restricted portion of the lumen may be about 6 mm, about 5 mm or about 4.5 mm.

The height of the projections may be selected such that when the ET tube is used as part of an intubation system also including a stylet or other tool, and the stylet/tool is inserted into the ET tube, at least part of the projections forms a light mechanical interference with a body of the stylet/tool. This can help to ensure suitable alignment of the stylet/tool within the ET tube. Furthermore, when the stylet has imaging capabilities, it can help to ensure that the optical axis of an imaging sensor of the stylet is suitably aligned within the ET tube. This suitable alignment may be an alignment in which an optical axis of an imaging sensor of the stylet is offset from a central longitudinal axis of the ET tube. This may reduce the risk of obscuration of the field of view of the imaging sensor e.g. by a distal tip portion of the ET tube. Such offset alignment may be achieved by suitably arranging the one or more projections within the ET tube. Accordingly, in some arrangements, the one or more projections may be arranged asymmetrically within the ET tube, or the height of the projections may vary asymmetrically—for example, one projection may have a maximum height that is larger than the maximum height of one or more other projections. Preferably, at least one projection is located along the axis of the longest longitudinal dimension of the tip of the ET tube. This can further assist in reducing the risk of obscuration of the field of view of the imaging sensor e.g. by a distal tip portion of the ET tube.

In some embodiments, the projections may have a maximum height (measured in a radial direction of the ET tube) that is in a range of from 0.5 mm to 3 mm, more preferably in a range of from 0.75 mm to 2 mm. Where there are multiple projections, in some arrangements the maximum height of the projections may be approximately identical for all projections. Alternatively in other arrangements the maximum height of the projections may vary in height.

In some embodiments, the projections may have a width (measured in a circumferential direction of the ET tube) in a range of from 0.5 mm to 3 mm wide, more preferably in a range of from 1 mm to 2 mm wide, e.g. about 1.5 mm wide.

In some embodiments, the projections may have a length (measured in a longitudinal direction of the ET tube) in a range of from 1 mm to 100 mm long, more preferably in a range of from 2 mm to 25 mm long, or from 5 mm to 15 mm long.

The projection(s) may be configured to increase the stiffness of at least a part of the endotracheal tube. For example, when the projection(s) are located in a distal tip portion of the ET tube, they may be configured to prevent buckling of the distal tip in response to applied axial loading, e.g. during movement of the patient or manipulation by the clinician. In particular, they may be configured to prevent such buckling under applied axial loads greater than or equal to the applied axial load required for yielding of the endotracheal tube main body. In other words, the endotracheal tube may be configured such that the main tube body buckles at a lower applied axial load than the distal tip portion of the device. This provides the technical advantage that during use of the endotracheal tube, when the tip is placed against an obstruction (e.g. within the trachea of a patient), the distal tip will brace against opposing walls of the trachea rather than buckling, thereby providing axial force/resistance feedback to the clinician and mitigating risk of tube obstruction and/or physical trauma to the patient. In some preferred arrangements, the ET tube may be configured such that the distal tip portion of the device resists buckling at applied axial loads of 20 N or more, more preferably 25 N or more or 30 N or more. In comparison, the main tube body may be configured such that it resists buckling at applied axial loads of 5 N or more, 6 N or more, 7 N or more, 8 N or more, 9 N or more, 10 N or more, or 15 N or more.

In some preferred embodiments, the largest dimension of the or each projection is a length of the projection as measured in a longitudinal direction of the ET tube. That is, as discussed above, the projections may comprise elongate ribs extending in a longitudinal direction of the ET tube. It may be advantageous for the largest dimension of the projection to be oriented to the long axis of the device as this can reduce the impact of the projections on the flow resistance to respiratory gases during use of the ET tube, as well as increasing flexural stiffness of the distal tip of the endotracheal tube.

Geometric arrangements as described above may be advantageous as they can reduce the risk of a stylet, or any other components intended to pass through the central lumen of the ET tube getting caught on the projections, or, where the shape of projections is configured to prevent the stylet or component protruding beyond the end of the ET tube during use, may suitably achieve this aim. In addition, use of a narrow elongate ramp shape can reduce negative impact on the flow resistance of the ET tube by the projections during use, thereby enabling ease of expiration of mechanically ventilated patients and ensuring minimal or no impact on ventilator settings as compared to conventional ET tube arrangements with a uniform bore profile.

As noted above, the internal projections project radially inwardly from an internal wall surface of a distal tip portion of the endotracheal tube. The internal wall surface may be an internal wall surface provided by a central lumen of the ET tube. The projections may be integrally formed with the internal wall surface. Alternatively, the projection may be disposed on, or otherwise attached to, the internal wall surface to thereby extend from the surface. Arrangements in which the projections are integrally formed with the internal wall surface of the tube may be preferred, as the risk of accidental detachment of the projections may be lower than arrangements in which the projections are provided as separate components attached to an internal wall surface of the tube. Accordingly, arrangements in which the projections are integrally formed with the internal wall surface of the tube may provide for improved patient safety in comparison to other arrangements.

The number of projections that the ET tube comprises is not particularly limited. In some arrangements, the endotracheal tube may comprise only a single projection. This can reduce manufacturing cost by minimising material needed in manufacture of the ET tube. In other arrangements, the ET tube may comprise two or more projections, three or more projections, or four or more projections. Where the ET tube comprises two or more projections, the projections may be substantially identical. Alternatively, some or all of the projections may differ in shape and/or size. In one particularly suitable arrangement, the ET tube may comprise at least three projections, wherein the projections are spaced at 90° intervals about a circumference of the ET tube. In other words, when viewed in a cross-section taken perpendicular to a longitudinal axis of the ET tube, the projections may be arranged at 90° , 180° and 270° about the circumference of the ET tube. Such an arrangement may provide for even further improved alignment of video stylets or other intubation aids within the endotracheal tube during use.

The or each projection may further comprise an axially facing detent surface. The axially facing detent surface may face towards a proximal end of the endotracheal tube. During use of the endotracheal tube in combination with a stylet or other intubation aid, the axially facing detent surface may engage with a distal end of said stylet or intubation aid to mitigate the risk of the stylet tip or intubation aid extending beyond the distal opening of the endotracheal tube, and thereby improve patient safety. This risk is elevated where softer tube materials are employed in ET tube manufacture where flexure and manual handling during use can change the overall tube length and can cause differential movement of the stylet within the tube, and so the provision of this axially facing detent surface may be particularly advantageous when provided in endotracheal tubes having a distal tip formed from a ‘soft’ material.

The axially facing detent surface may be provided by a lip or shoulder portion projecting radially inwardly towards the centre of the bore/lumen of the endotracheal tube. The lip/shoulder may be arranged at a distal end of the or each projection. Such lip/shoulder portions may prevent the passage of the distal tip of a stylet or other intubation aid beyond the lip/shoulder.

Further features relating to the endotracheal tube will now be discussed. These features are equally applicable to the first and/or second aspects of the invention.

Various dimensions of the ET tube may depend on its intended use. For example, depending on the intended use of the tube, the tube may be manufactured to have a predetermined nominal internal diameter. In preferred arrangements, an internal diameter of the ET tube (measured as a diameter of the central lumen of the tube) may be in a range of from 5 mm to 10 mm, more preferably in a range of from 6 mm to 8 mm. For example, the internal diameter of the ET tube may be 6.5 mm, 7 mm, 7.5 mm, or 8 mm.

The wall thickness of the ET tube body (the ‘main tube wall thickness’) may be in a range of from 1 mm to 2 mm, more preferably 1.2 to 1.7 mm. For example, the main tube wall thickness may be 1.2 mm or more, 1.3 mm or more, 1.4 mm or more 1.5 mm or more, or 1.6 mm or more. It may be 1.6 mm or less, 1.5 mm or less, 1.4 mm or less, or 1.3 mm or less. The main tube wall thickness may be substantially constant along the length of the ET tube. Alternatively, it may vary along the length of the ET tube. The preferred wall thicknesses identified above may be average thicknesses determined by taking a mean value of at least 2 sample values for the main tube wall thickness measured at different locations along the length of the ET tube.

As discussed above, the ET tube body comprises a flexible hollow tube—in other words, it comprises a generally tubular body which comprises a tube wall having an inner and an outer wall surface. In some embodiments, the ET tube body comprises a single integral component. In some alternative embodiments, the ET tube body may be formed from two or more components joined together. In one preferred arrangement, the ET tube body comprises a main body portion and a distinct distal tip portion which are separate components, i.e. components that are not integrally formed. These separate components may accordingly be subsequently joined together to form the ET tube body. Each of the components may be made using a moulding process. In such an arrangement, the main body portion and the distal tip portion may be joined in any suitable manner. For example, they may be joined by friction fit alone, or by an adhesive, welding or by mechanical fixing means or a combination of methods. For example, in one preferred method, the distal tip portion may be first glued to the main body via a socket/lap joint and is subsequently RF welded to ensure secure attachment.

The material(s) from which the ET tube is manufactured are not particularly limited and the person skilled in the art will be aware of a range of materials which would be suitable for use in such an application. As noted above, the ET tube body may be polymeric. This allows suitable bending flexibility and ease of manufacture to allow incorporation of the helically wound, meshed or braided reinforcement structure to be embedded therein. One example of a suitable material for the ET tube body is PVC.

The present invention may allow for use of softer grade materials than used in conventional ET tubes, due to the greater resistance to buckling of the distal tip provided by the projections. One suitable measure for the ‘softness’ of a material is Shore A Hardness. Shore A hardness is measured according to the ASTM D2240 type A testing standard. It has been found that endotracheal tubes according to the present invention can suitably include a part (e.g. the distal tip portion and/or the main body portion) having a Shore A Hardness of 75 or less, for example 70 or less or 65 or less, in some cases as low as 60. This is in comparison to conventional endotracheal tubes which typically have a Shore A hardness of 78 or more. The use of a softer grade material for part of the endotracheal tube can reduce the risk of injury to a patient during use. However, this increased softness should be balanced with the desirability to provide a distal tip which can provide a user (e.g. a clinician) with sufficient tactile feedback during use (e.g. on impaction of the tip with an internal structure of a patient. In this regard, it is considered that endotracheal tubes according to the present invention may suitably include a part (e.g. the distal tip portion) having a Shore A Hardness in a range of from 60 to 75. It has been found that this range can provide a good balance of softness and physical feedback to a clinician during use.

Where the ET tube body comprises a main body portion and a distinct distal tip portion which are separate components, these components may be formed from similar or dissimilar materials. In one preferred arrangement, the distal tip portion is formed from a material having greater bending flexibility as compared with the main body portion of the ET tube. This can help to prevent unwanted injury to a patient during an intubation procedure by allowing greater bending flexibility of the articulating portion of the tube. In one embodiment, the main body of the ET tube may be made from a first material selected from a PVC, thermoplastic elastomer, or silicone material, and the distal tip portion may be made from a different material. For example, the distal tip portion may be made from a second material selected from a PVC, thermoplastic elastomer, or silicone, that is different to the first material.

The proximal end of the ET tube may be attached to a connector for connection of the ET tube to ventilation apparatus (e.g. after an intubation procedure has been completed). Where such an ET tube connector is present, it may be removable: this can allow the ET tube to be cut to length after insertion, and then subsequently replaced to allow connection of the ET tube to ventilation apparatus.

The distal end of the ET tube may have a bevelled tip, to aid in insertion of the tube between the vocal cords of the patient. The distal end of the tube may be open at its distal end. The distal end of the tube may also have a subsidiary opening to provide an alternate gas passage in the case of occlusion of the main opening, e.g. an opening known as a Murphy eye formed in a sidewall of a tip portion of the tube. In some arrangements, the ET tube may have a single such opening formed in a sidewall of a tip portion of the tube, i.e. it may have a single Murphy eye. Alternatively, the ET tube may have multiple such subsidiary air openings, for example two Murphy eyes. The openings may be formed in opposing sidewalls of a distal tip portion of the ET tube. Where there are multiple openings, they can be smaller whilst retaining the same total flow area for the passage of gas in comparison to typical known tubes having only one Murphy eye. The advantage of providing multiple, smaller openings or Murphy eyes is that it can prevent a stylet tip from passing through or catching in these openings.

The unintended consequence of such openings in the sidewall of the tip is the formation of weakened portions of the tip which may provide an increased risk of the tip collapsing or folding under mechanical loading. Accordingly, by providing appropriately positioned projections as previously described, it may be possible to compensate for the weakening of the tip by said openings, thereby mitigating the risk of the tube collapse or folding under mechanical loading. This is because the projections can act as strengthening members, enable the choice of softer grades of PVC than would otherwise be necessary to prevent tube collapse or folding. The employment of a softer grade of material is conducive to limit physical trauma which can occur during the intubation process. In one preferred arrangement, at least one internal projection may be arranged to oppose an opening formed in a sidewall surface of the distal tip of the tube. That is, said projection may be arranged at approximately 180° about a circumference of the ET tube with respect to the opening. In a further preferred arrangement, three projections are provided, wherein the projections are spaced at 90° intervals about a circumference of the ET tube, and wherein at least one of the projections opposes an opening formed in a sidewall surface of the distal tip of the tube. In other words, when viewed in a cross-section taken perpendicular to a longitudinal axis of the ET tube, the opening may be arranged at 0/360° , and the projections may be arranged at 90° , 180° and 270° about the circumference of the ET tube.

Where the bending flexibility is defined by the peak torque recorded on a torque gauge (e.g. H10S digital torque meter) on actuation of a control member (e.g. rotatable control wheel) of an articulated stylet (e.g. of the type described in GB2563567B) disposed within a central lumen of the ET tube during flexion/retroflexion of the ET tube by control of the articulated stylet, the endotracheal tube may be configured to bend (flex or retroflex) at an applied torque of 1 Nm or less, for example 0.9 Nm or less, or 0.8 Nm or less to reach an articulation angle of 50° , the articulation angle being measured as the angle of deflection of the distal end of the ET Tube away from a neutral position. In preferred arrangements, the endotracheal tube may be configured to bend at an applied torque of less than about 0.75 Nm, for example less than 0.6 Nm, less than 0.5 Nm, less than 0.4 Nm, less than 0.35 Nm or less than 0.3 Nm to reach an articulation angle of 50° . This force may be measured at the control member of the stylet.

Allowing bending of the ET tube at applied forces/torques having these values can help to facilitate ease of manipulation by the user. In particular, it has been found that if torques greater than 1 Nm are required to flex or retroflex the ET tube tip to reach an articulation angle of 50° , then this may undesirably compromise usability of the ET tube. This may be achieved by appropriate selection of various properties of the endotracheal tube, including e.g. appropriate selection of materials and dimension of the ET tube, appropriate configuration of internal projections provided in the distal tip portion, if present, and/or appropriate selection of characteristics of the helically wound, meshed or braided reinforcement structure.

The endotracheal tube may be configured to resist collapse at pressures up to and including 300 cmH2O or higher (29420 Pa or higher), for example, it may be configured to resist collapse at pressures of 310 cmH2O or more, 320 cmH2O or more, 330 cmH2O or more, 340 cmH2O or more, 350 cmH2O or more, or 400 cmH2O or more. In some embodiments, the endotracheal tube may be configured to resist collapse at pressures up to 500 cmH2O. This may be achieved by appropriate selection of various properties of the endotracheal tube, including e.g. appropriate selection of materials and dimension of the ET tube, appropriate configuration of internal projections provided in the distal tip portion, if present, and/or appropriate selection of characteristics of the helically wound, meshed or braided reinforcement structure. By providing such an arrangement, a significant reduction in patency can be avoided under most or all standard operation conditions, according to standards set out in BS EN ISO 5361:2016, Clause 5.5.4/Annex C. The inventors note that the highest recorded in-use pressure applied to an endotracheal tube similar to the present invention is around 210 cmH2O. Accordingly, by providing a tube which is able to resist collapse even at much higher pressures than this, patient safety during use of the ET tube can be improved.

The endotracheal tube may further comprise an inflatable cuff provided at or near a distal end region of the endotracheal tube. The inflatable cuff may be configured for inflation during an intubation procedure once the ET tube has been positioned in a desired location, to hold the tube in the correction position to protect from pulmonary aspiration and permit positive pressure ventilation of the patient. Where the tube has an inflatable cuff, this may be connected to an inflation line through which air may be pumped to inflate the cuff. At least a portion of the inflation line may be recessed into the wall of the ET tube. The inflation line may be configured for connection to an external air supply in a conventional manner. Provision of such a cuff is a standard feature of many well-known ET tube designs, and as such, the size and shape of the cuff is not particularly limited. Furthermore, the material which the inflatable cuff is made from is not particularly limited and the skilled person will be well aware of a number of suitable materials which could be used for this purpose.

Provision of an ET tube which has a minimum predetermined resistance to collapse may be particularly preferred for ET tubes which have an inflatable cuff, because of the risk of accidental over-inflation of the cuff, leading to higher than expected pressure being applied to the ET tube, which may consequently result in an increased risk of ET tube collapse during an intubation procedure. Accordingly, in one preferred embodiment, the endotracheal tube comprises an inflatable cuff provided at a distal end region of the endotracheal tube, and the endotracheal tube is configured to resist collapse at pressures up to and including 300 cmH2O (or more—see ranges set out above) at a region underlying the inflatable cuff. The provision of reinforcement of the ET Tube up to the distal end of the cuff facilitates the strengthening of the tube to resist collapse at elevated cuff inflation pressures.

Preferably the ET tube does not itself comprise an actuating mechanism for bending of the ET tube during an intubation procedure. Rather, it is preferred that the ET tube is configured to receive a stylet which is inserted into the lumen of the hollow ET tube. The ET tube may be configured to receive a stylet of the type described in GB2563567B, which is herein incorporated by reference.

Accordingly, in a third aspect, the present invention provides an intubation system comprising an ET tube according to the first aspect, or an ET tube according to the second aspect, and a stylet for guiding an endotracheal tube during intubation.

Preferably, the stylet comprises a body having a pivotable tip located at a distal end of the body, the pivotable tip moveable in two opposing directions from the longitudinal axis of the distal end of the stylet body, and a control mechanism for controlling the pivot angle of the pivotable tip.

As discussed above, the ET tube may comprise a connector at the proximal end. The connector may be configured for attachment to a corresponding connector provided on the stylet. For example, the stylet may comprise a plug connector portion adapted to provide a plug fit connection to the connector of the ET tube. This arrangement may prevent relative longitudinal movement of the stylet with respect to the ET tube (e.g. during an intubation procedure). It may additionally provide for a predetermined alignment of the stylet and the ET tube, to (a) ensure that the stylet does not protrude from the distal end of the ET tube during intubation, and/or (b) ensure that a bending portion of the stylet can be aligned with a bending portion of the ET tube.

In particular where the stylet intended for use with the ET tube is a ‘pivot-end’-type or articulated stylet, the ET tube and stylet may be respectively configured to allow for connection of these two elements to give a predetermined relative alignment in which an articulation or pivot point (e.g. hinge) of the ‘pivot-end’-type/articulated stylet generally aligns with a predetermined bending region of the endotracheal tube, when the stylet and ET tube are connected together for use. ‘Generally aligns with’ can be understood to mean that the pivot point may be located at a distance of ±5 mm from the location of the predetermined bending region of the ET tube. Features of the ET tube and stylet which may be selected to allow this predetermined relative alignment include the length of the ET tube, the location of the predetermined bending region along the length of the ET tube, the length of the stylet, the location of the pivot point along the length of the stylet, and the shape and/of length of respective connectors of the ET tube and the stylet. In some arrangements, the alignment of the pivot point of the stylet and bending region/local flexure point of the ET tube may be facilitated by provision a detent on the connector portion of the stylet which ensures a consistent datum with the endotracheal tube, when said connector portion is engaged with the ET tube connector.

As discussed above, provision of internal projections within the endotracheal tube may aid cooperative use of the endotracheal tube and a stylet, or may aid cooperative use of the endotracheal tube and one or more further instruments intended for use with the ET tube—in particular, instruments which may be passed through the central lumen of the ET tube during use. Provision of such alignment features is particularly advantageous in intubation systems comprising a stylet having imaging capabilities (i.e. when the stylet is a video stylet), as this can help to ensure that obscuration of the field of view of the stylet does not occur during use.

The intubation system of the second aspect can be used to perform an intubation process by a) inserting the stylet into the ET tube, b) inserting the stylet and ET tube into the airway of a patient, c) visualising the airway of the patient, e.g. via an image acquisition device provided on the stylet, d) guiding the ET tube and stylet through the vocal cords of the patient into the trachea, and e) removing the stylet from the ET tube. Such a system can offer increased ease of intubation, in particular for patients with difficult airways. Accordingly, a further aspect of the present invention is an intubation method using the endotracheal tube or intubation system disclosed herein.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 is a perspective view of an intubation system according to one embodiment of the present invention, with the endotracheal tube and stylet shown separately.

FIGS. 2(a) and (b) respectively show side and plan views of the intubation system shown in FIG. 1, with the stylet connected to the endotracheal tube for use.

FIG. 3 shows a schematic view of the intubation kit of FIGS. 1 and 2 in use in an intubation process.

FIGS. 4 (a) and (b) are cross-sectional views of an endotracheal tube 1′ according to an embodiment of the invention.

FIG. 5 is a schematic view of the same embodiment as shown in FIG. 4(a) and (b) (not to scale).

FIG. 6 is a schematic detail view of the region indicated in FIG. 4(b).

FIG. 7 shows a perspective view of a distal tip portion of an endotracheal tube according to one embodiment of the present invention.

FIG. 8 shows a cross-section view of the distal tip portion of FIG. 4.

FIGS. 9(a) and (b) show further cross-sectional views of the distal tip portion of FIG. 7, taken in sections A-A and B-B as indicated in FIG. 5.

FIG. 10 shows a perspective view of a distal tip portion of an endotracheal tube according to a further embodiment of the present invention.

FIG. 11 shows a cross-section view of the distal tip portion of FIG. 10.

FIGS. 12(a) and (b) show further cross-sectional views of the distal tip portion of FIG. 10, taken in sections A-A and B-B as indicated in FIG. 11.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

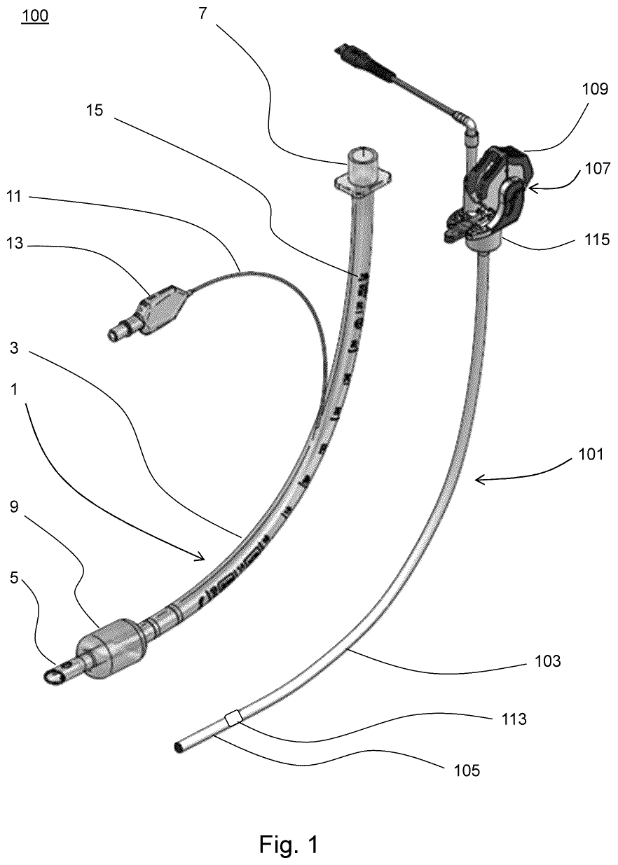

FIG. 1 is a perspective view of an intubation system 100 according to one embodiment of the present invention, with the endotracheal tube and stylet shown separately.

The endotracheal tube 1 comprises a flexible hollow tube body with a distal and a proximal end. Distal and proximal are here described in relation to the use of the endotracheal tube, with the proximal end being the end of the tube which is typically held by an operator during use in a process of intubation. The distal end of the endotracheal tube is the end which, in use, may be inserted into a patient's airway to assist in an intubation process. Whilst not easily visualised in FIG. 1, the endotracheal tube body comprises a main body portion 3 and a distinct distal tip portion 5 having a bevelled end. These are separate components which are joined together to provide the ET tube body—here, the components are conveniently joined by a combination of adhesive and RF welding. The main body of the ET tube is made from a first type of PVC, and the distal tip portion is made from a second type of PVC, Thermoplastic Elastomer or silicone. The material of the distal tip portion is selected to have greater bending flexibility as compared to the first type of PVC.

The endotracheal tube further comprises a connector 7 provided at the proximal end. This connector is configured for connection of the ET tube to ventilation apparatus after an intubation procedure has been completed. This connector is also configured for connection to a corresponding connector provided on the stylet, as will be discussed in greater detail below. The connector is a removable connector (i.e. separate component) which fits into the main tube. It is preferably attached via a ‘dry’ joint (i.e. not using adhesive) which is achieved by stretching the tube's proximal end over a cylindrical portion of the connector—i.e. the connection between the removable connector and the ET tube main body is a fiction fit connection.

The endotracheal tube comprises an inflatable cuff 9 and corresponding inflation line 11 with pilot balloon 13.

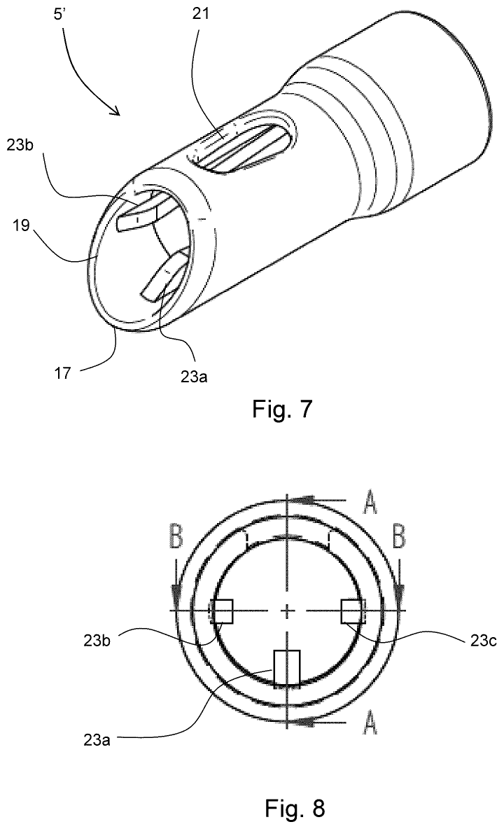

The ET tube includes two Murphy eyes formed in the distal tip portion 5 of the tube, on opposing sides of the tube, each Murphy eye being arranged at 90° with respect to an axis of the longest longitudinal dimension of the distal tip portion. These openings provide alternative flow paths for air in the cause of occlusion of the main outlet of the tube. The Murphy eyes are sized to limit or prevent protrusion of a stylet through the openings. These are not essential and may not be provided in some embodiments. In other embodiments, as will be discussed below in relation to FIG. 7-12, a single Murphy eye is provided.

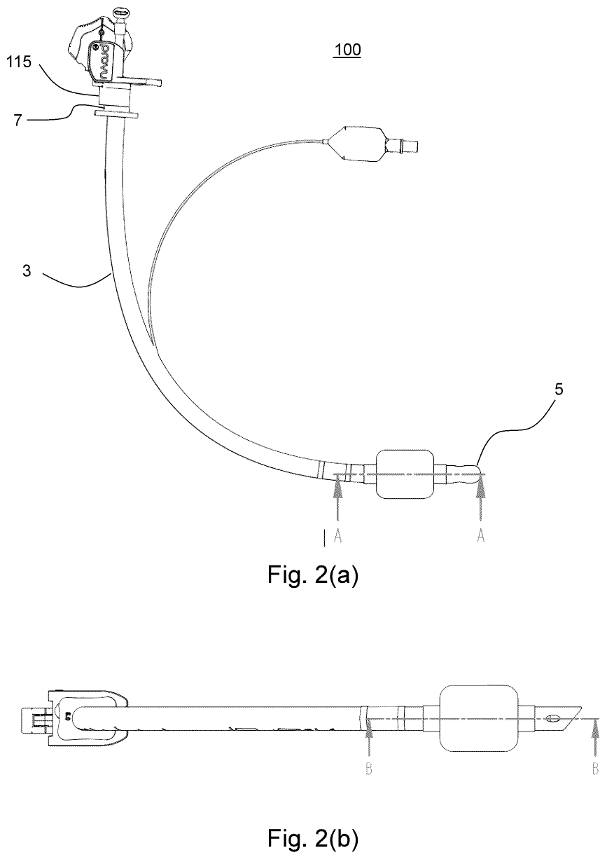

The distal tip portion of the tube also comprises internal projections, however these are not visible in FIGS. 1 and 2. Examples of suitable configuration of internal projections of an endotracheal tube according to the present invention are described below in relation to FIGS. 7-12 which illustrate distal tip portions of further embodiments.

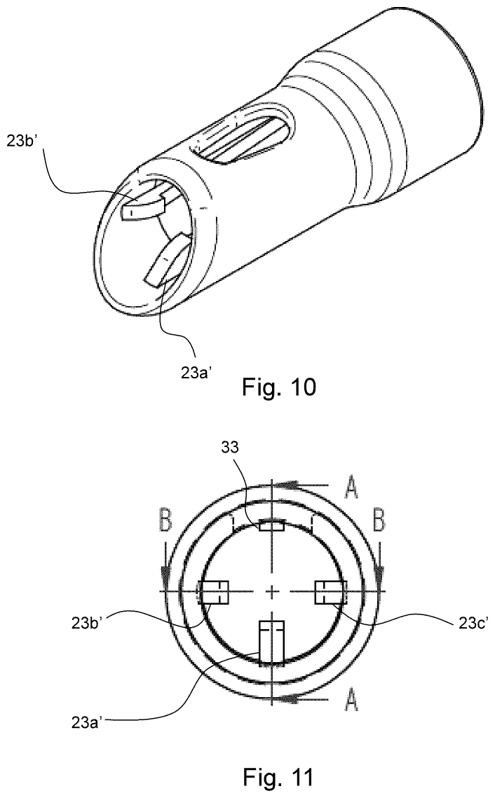

The endotracheal tube body comprises a polymeric material (PVC, as discussed above) comprising a helically wound reinforcement structure embedded therein. In the present case, the helically wound reinforcement structure is formed from a single helically wound stainless steel filament, although the reinforcement structure is not visible in FIG. 1 or 2. FIG. 10 illustrates a further embodiment of an endotracheal tube according to the present invention comprising a similar reinforcement structure, which is discussed in further detail, below.

Considering the stylet 101, this has a stylet body 103 with a distal and a proximal end. As with the ET tube, the proximal end is the end of the stylet which is typically held by an operator during use in a process of intubation, and the distal end of the stylet is the end which, in use, may be inserted into a patient's airway to assist in an intubation process. The stylet has a pivotable tip 105 located at the distal end of the stylet body, and an actuator 107 attached at the proximal end of the stylet body. The actuator is here conveniently manipulated by the operator using a thumb-pad 109 on a dial which rotates around an axle. The pivotable tip is affixed to the stylet body 103 at a pivot hinge 113 which allows movement of the pivotable tip in a plane. The pivotable tip has imaging capabilities provided by an imaging sensor (not shown) located at a distal end of the pivotable tip. The stylet has a connector 115 located at the proximal end of the stylet, formed integrally with the body/retaining housing of the actuator. The connector here is formed as a receiving socket or plug portion which forms a plug-fit connection with the ET tube connector 7. Other features of the stylet are discussed in further detail in GB2563567B.

FIGS. 2(a) and (b) respectively show side and plan views of the intubation system shown in FIG. 1, with the stylet connected to the endotracheal tube for use. It can be seen from this figures that the connector 7 of the endotracheal tube and the connector 115 of the stylet are configured to allow for plug-type/socket-type connection, with the stylet connector 7 providing a female socket portion into which the (male) endotracheal tube connector can be inserted. A detent is provided within the female socket portion of the stylet connector (not visible). This ensures a consistent datum with the endotracheal tube, when the connectors are engaged for use. This arrangement helps to prevent relative longitudinal movement of the stylet with respect to the ET tube (e.g. during an intubation procedure), and also provides a predetermined alignment of the stylet and the ET tube, to (a) ensure that the stylet does not protrude from the distal end of the ET tube during intubation, and (b) ensure that the pivot hinge of the stylet can be aligned with a bending portion of the ET tube.

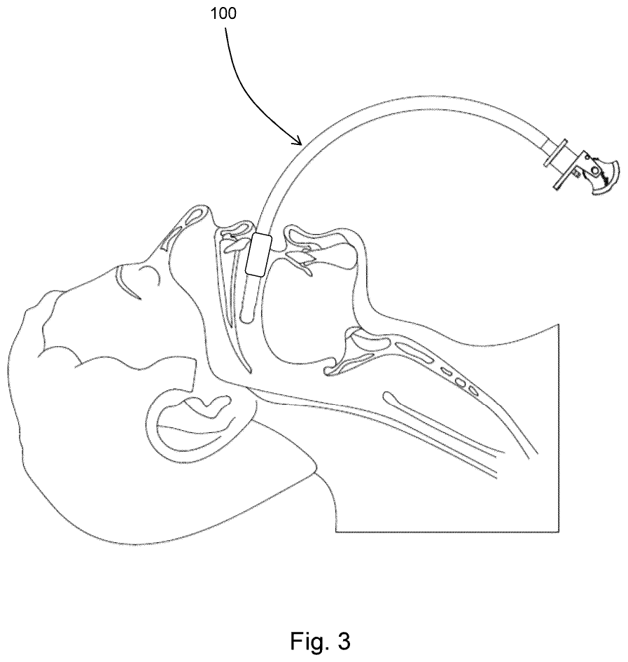

The intubation kit 100 shown in FIGS. 1 and 2 typically finds use in intubation procedures. FIG. 3 shows a schematic view of the intubation kit 100 of FIGS. 1 and 2 in use in an intubation process. As can be seen from the drawing, the ET tube body 1 is generally flexible along its length and can curve to fit the patient's airway. The additional flexibility provided by the spaced locally thinned circumferential wall portions provided at the distal end region of the endotracheal tube allows for ease of manipulation of the distal end of the ET tube using the pivotable stylet tip, which is controlled by a user using the actuator of the stylet which remains outside of the patient's body during use. A user can more easily guide the ET tube into the desired location during the intubation procedure.

The kit can be used to perform an intubation process, including steps of a) inserting the stylet into the ET tube, b) inserting the stylet and ET tube into the airway of a patient, c) visualising the airway of the patient, d) guiding the ET tube and stylet through the vocal cords of the patient into the trachea of the patient, and e) removing the stylet from the ET tube.

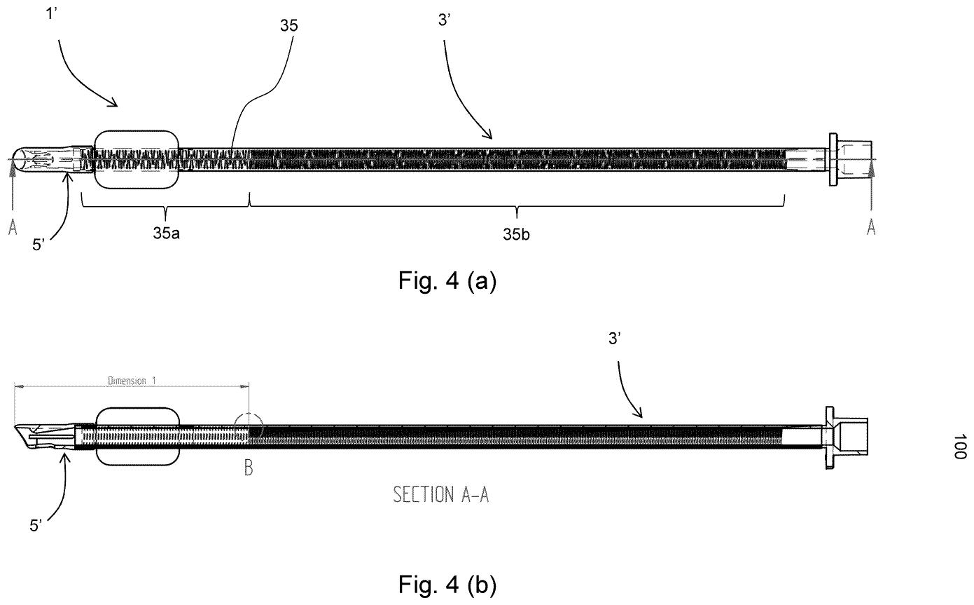

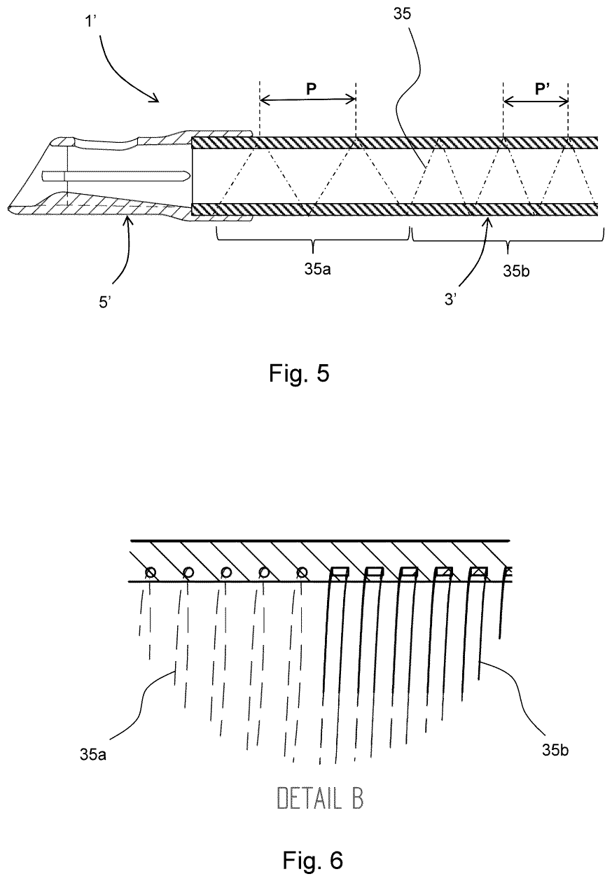

FIGS. 4(a) and (b) are cross-sectional views of an endotracheal tube 1′ according to an embodiment of the invention. FIG. 5 is a schematic view of the same embodiment (NB: length scales in schematic FIG. 5 do not correspond to those shown in FIGS. 4(a) and (b) but are selected to clearly demonstrate various features discussed below). FIG. 6 is a schematic detail view of the region of the endotracheal tube 1′ indicated in FIG. 4(b).

Similarly to the arrangement shown in FIG. 1, the endotracheal tube body comprises a main body portion 3′ and a distinct distal tip portion 5′ having a bevelled end. The distal tip portion comprises internal projections: the structure and shape of the distal tip portion of this embodiment is discussed in further detail below in relation to FIG. 7-9.

In this embodiment, the endotracheal tube body comprises a polymeric material comprising a helically wound reinforcement structure 35 embedded therein. The reinforcement structure extends along a major proportion of the ET tube body but does not extend within a distal tip portion of the tube. The lines representing the helically wound reinforcement structure 35 are shown as dashed in some figures (e.g. FIG. 5) to represent that the structure is embedded within the sidewalls of the endotracheal tube body. It can be seen that the pitch P of the helically wound reinforcement structure varies along the length of the main body portion of the tube—this is most clearly shown in schematic FIG. 5. The reinforcement structure can be divided conceptually, into two parts: a first helically wound filament portion 35a, and a second helically wound filament portion 35b. For ease of manufacture, these portions are provided as two distinct and separate filaments arranged sequentially along the length of the endotracheal tube, although it is also contemplated that they could be provided as portions of a single continuous filament. The pitch P of the first helically wound filament part 35a is greater than the pitch P′ of the second helically wound filament part 35b. For both parts 35a and 35b, the pitch of the helical winding is in a range of from 0.75 mm to 3 mm. Accordingly, it can be seen that the pitch of helical winding (and corresponding also the relative volume proportion of the filament(s) forming the reinforcement structure within the endotracheal tube) varies along the length of the endotracheal tube, in a step-wise manner.

In this embodiment, the cross-sectional shape of the filament(s) forming the reinforcement structure also varies along the length of the endotracheal tube. The first filament portion 35a has a substantially circular cross-sectional shape in a cross-section taken perpendicular to the direction of extension of the filaments. The second filament portion 35b has a flattened (substantially rectangular) cross-sectional shape in the same cross section.

It has been found that this arrangement for a reinforcement structure is particularly preferred, as it can ensure resistance to crushing and kinking of the endotracheal tube in a proximal region of the tube (e.g. a region which in use, extends from the upper airway to outside the patient's body), whilst allowing a more flexible cross-section in the distal portion of the tube to ensure optimal flexibility of the tube in this region whilst providing adequate resistance to collapse.

In other embodiments, the specific form and structure of both parts 35a and 35b may be varied depending on the nominal internal diameter of the endotracheal tube, e.g. in line with the details set out in the tables below. In the tables below, the term “spring” is used to refer to a helical coil of reinforcing filament. The first helically wound filament portion 35a is referred to as the distal reinforcement section. This is a filament having a substantially circular cross-sectional shape, and so the size of the filament is given as a diameter. The second helically wound filament portion 35b is referred to as the proximal reinforcement section. This is a filament having a substantially rectangular cross-sectional shape, and so the size of the filament is given as a combination of width and thickness of the filament.

| | | | | |
|---|---|---|---|---|
| ET Tube | | | Wire cross | |
| nominal | | Spring | sectional | |
| internal | Spring | Outer | diameter - | |
| diameter | Length | Diameter | circular cross | Pitch |
| (mm) | (mm) | (mm) | section (mm) | (mm) |

Distal Reinforcement Section

| | | | | |
|---|---|---|---|---|
| 65 | 64 | 7.4 | 0.3 | 1.2 |
| 70 | 70 | 7.9 | 0.3 | 1.2 |
| 75 | 72 | 8.4 | 0.3 | 1.2 |
| 80 | 72 | 8.9 | 0.3 | 1.3 |

| | | | | | |
|---|---|---|---|---|---|
| ET Tube | | | | Wire | |
| nominal | | Spring | Wire Width - | Thickness- | |
| internal | Spring | Outer | rectangular | rectangular | |
| diameter | Length | Diameter | cross | cross | Pitch |
| (mm) | (mm) | (mm) | section (mm) | section (mm) | (mm) |

Proximal Reinforcement Section

| | | | | | |
|---|---|---|---|---|---|
| 65 | 285 | 7.4 | 0.5 | 0.25 | 1.1 |
| 70 | 300 | 7.9 | 0.5 | 0.25 | 1.1 |
| 75 | 310 | 8.4 | 0.5 | 0.25 | 1.1 |
| 80 | 315 | 8.9 | 0.5 | 0.25 | 1.3 |

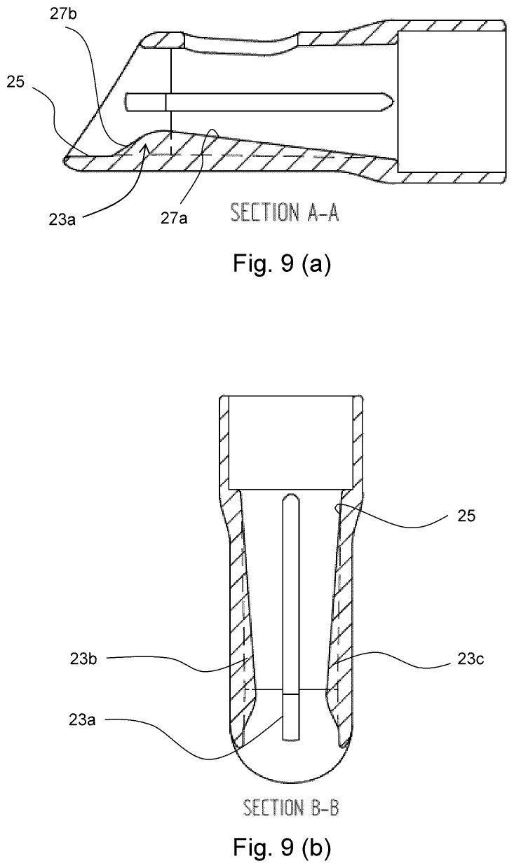

Further details relating to the configuration of a distal tip portion of endotracheal tube according to the present invention will now be discussed in relation to FIG. 7-12. FIG. 7 shows a perspective view of a distal tip portion of an endotracheal tube according to one embodiment of the present invention, with FIGS. 8, 9(a) and 9(b) showing various cross-sectional views of the distal tip portion shown in FIG. 7.

The distal tip portion 5′ in FIG. 7-9 is a discrete component configured for connection to a main body portion of an endotracheal tube, e.g. by use of an adhesive, RF welding, or a combination thereof. It has a bevelled tip 17 to aid in insertion of the tube between the vocal cords of the patient, which provides the longest longitudinal dimension of the distal tip potion. The distal tip portion is open at its distal end at a main opening 21, and further comprises a subsidiary opening (Murphy eye) 21 which is formed in a sidewall surface of the distal tip potion. This Murphy eye provides an alternate gas passage in the case of occlusion of the main opening.

The distal tip portion comprises three internal projections 23a, b, c. Each of these internal projections is formed as an elongate rib having an extension direction which is substantially parallel to a longitudinal axis of the endotracheal tube.

The projections 23a, b, and c have generally similar sizes and shapes, however projection 23a has a slightly larger maximum height than projections 23b and 23c, as can be seen in FIG. 8. The precise height of each projection may vary depending on the internal diameter of the ET tube. Some examples of suitable projection heights are set out in the table below, where ID is the nominal internal diameter of the endotracheal tube, the ‘major riblet’ corresponds to projection 23a, and the ‘minor riblet’ corresponds to projections 23b, 23c:

| ID (mm) | Major Riblet Height (mm) | Minor Riblet Height (mm) |
|---|---|---|
| 6.50 | 1.50 | 0.75 |
| 7.00 | 2.00 | 1.00 |
| 7.50 | 1.50 | 0.75 |
| 8.00 | 2.00 | 1.00 |

This arrangement can allow the projections to engage with a stylet or other intubation device within the ET tube to provide an offset alignment of the stylet/intubation device with respect to the ET tube. Where the stylet has imaging capabilities, this can reduce the risk of obscuration of the field of view of an imaging sensor of the stylet.

As best seen in FIG. 8, when viewed in a cross-section taken perpendicular to a longitudinal axis of the ET tube, the projections are arranged at 90° , 180° and 270° about the circumference of the ET tube, when the Murphy eye 21 is taken to be arranged at 0/360° . This specific arrangement has been found to be advantageous as it allows at least one projection to be located opposite the Murphy eye & can help to compensate for any weakening of the distal tip portion resulting from provision of the Murphy eye, thereby mitigating the risk of the tube collapse or folding under mechanical loading. In this way, projections 23a, b, and c act as strengthening members.

The profile of the projections is best seen in FIGS. 9(a) and 9(b). Here it can be seen that the projections 23a, b, c project radially inwardly from an internal wall surface 25 of a distal tip portion of the endotracheal tube. The projections 23a, b, c are tapered along their extension direction. That is, the height of the internal projections (measured in a radial direction of the ET tube) varies along the extension direction in a tapered manner. In this embodiment, the projections comprise a first portion which is tapered in a first direction, and a second portion which is tapered in a second direction: specifically, a proximal portion of the projections is tapered such that the height of the proximal portion decreases towards the proximal end of the ET tube, and a distal portion of the projections is tapered such that the height of the distal portion decreases towards the distal end of the ET tube. The maximum height of the projections is thereby provided at a point intermediate the proximal and distal portions of the projections. As a result of this tapering, the projections comprise first and second ramp surfaces e.g. projection 23a comprises a first ramp surface 27a provided by a proximal portion of the projection, and a second ramp surface 27b provided by a distal portion of the projection. Projections 23b and 23c analogously comprise first and second ramp surfaces. This geometric arrangement has been found to provide particularly good stylet/intubation device alignment during use of the ET tube, whilst minimizing the impact of the projections on the flow of respiratory gases during use of the ET tube.

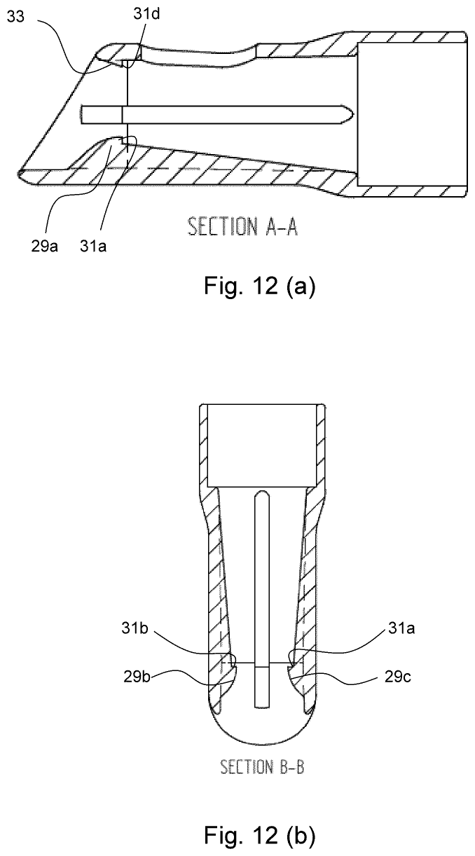

FIG. 10 shows a perspective view of a distal tip portion of an endotracheal tube according to a further embodiment of the present invention, with FIGS. 11, 12(a) and 12(b) showing various cross-sectional views of the distal tip portion shown in FIG. 10. The overall arrangement of this embodiment is generally similar to that of the embodiment discussed above, except that in this embodiment, each projection 23a′, 23b′, 23c′ further comprises a shoulder portion 29a, b, c projecting radially inwardly towards the centre of the bore/lumen of the endotracheal tube—this is best seen in FIGS. 12(a) and (b). Each shoulder portion provides an axially facing detent surface 31a, b,c, which faces towards a proximal end of the endotracheal tube.

An additional projection 33 is also provided in this embodiment as compared to the embodiment of FIG. 7-9. This additional projection extends from the internal wall surface of the distal tip adjacent to the Murphy eye. The height of the additional projection is substantially smaller than that of the three other projections (less than 50% of the height of the other projections), thereby allowing for offset alignment of a stylet/intubation aid as discussed above in relation to the previous embodiment. The primary purpose of this projection is to provide an additional axially facing detent surface 31d.

During use of the endotracheal tube in combination with a stylet or other intubation aid, the axially facing detent surfaces 31a, b, c, d may engage with a distal end of said stylet or intubation aid to mitigate the risk of the stylet tip or intubation aid extending beyond the distal opening of the endotracheal tube, and thereby improve patient safety.

The arrangements shown and described here provides a number of technical advantages over known endotracheal tubes and intubation systems. Specifically, it has been found that suitable flexure of the ET tube can be achieved in response to a predetermined bending force as the endotracheal tube is configured to bend at an applied actuation torque of <0.75 Nm to reach an articulation angle of 50°: this facilitates ease of manipulation by the user, as the longitudinally spaced locally thinned circumferential wall portions act as a bending portion or local flexure at the distal end of the tube. Additionally, the resistance of the ET tube to collapse during use is increased in comparison to known arrangements, with the endotracheal tube being configured to resist collapse at pressures up to and including 500 cmH2O or higher (29420 Pa or higher), to avoid a significant reduction in patency during standard operation, in line with standards set out in BS EN ISO 5361:2016, Clause 5.5.4/Annex C.

Furthermore, these arrangements can use a suitably soft material to reduce risk of injury to a patient (e.g. a material having a Shore A hardness of 75 or less), but where the tip still provides suitable resistance to buckling under axial loading, e.g. wherein the distal tip is able to resist buckling at applied axial loads of 20 N or more. Some experimental data was obtained showing required axial force to buckle various ET tubes according to the present invention—see table below. From this table it can be seen that regardless of the nominal internal diameter of the ET tube, and despite use of a material having a Shore A hardness of only 75, it was possible to provide ET tube having a distal tip portion resistant to buckling under applied axial loads of 22 N or more. The main tube body was allowed to have greater flexibility (resistance to buckling under applied axial loads of only 7.4 N or more):

| | | | | Minimum |
|---|---|---|---|---|
| | | | | bend |
| ET Tube | Axial force to | Axial force to | Tip Material | radius of |
| Diameter | buckle main | buckle distal | Hardness | main tube |
| (mm) | tube body (N) | tip portion (N) | (Shore A) | (mm) |
| 8 | 13.5 | 37 | 75 | 7 |
| 7.5 | 9.4 | 31 | 75 | 7 |
| 7 | 8.6 | 29 | 75 | 7 |
| 6.5 | 7.4 | 22 | 75 | 7 |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

What is claimed is:

1. An endotracheal tube having a body comprising a flexible hollow tube with a distal end for insertion into a patient's trachea during intubation, and an opposite proximal end, wherein: the endotracheal tube body comprises a polymeric material comprising a helically wound, meshed or braided reinforcement structure embedded therein, the helically wound, meshed or braided reinforcement structure being formed from one or more filaments, and wherein one or more properties of the reinforcement structure vary along the length of the endotracheal tube such that the bending flexibility and/or the mechanical strength of the endotracheal tube varies along the length of the endotracheal tube.

2. The endotracheal tube according to claim 1 wherein the material, the cross-sectional shape or area, and/or the relative volume proportion of the filament(s) forming the reinforcement structure vary along the length of the endotracheal tube.

3. The endotracheal tube according to claim 1 wherein the largest dimension of the or each filament in a direction perpendicular to the longitudinal extension of the filaments is in a range of from 0.1 to 1 mm.

4. The endotracheal tube according to claim 1 wherein the reinforcement structure comprises one or more helically wound filaments, and wherein the pitch of the helical winding of the filament(s) is in a range of from 0.75 mm to 5 mm, optionally wherein the pitch of the helical winding of the filament(s) varies along the length of the endotracheal tube.

5. The endotracheal tube according to claim 4 wherein a first helically wound filament portion is arranged distally of a second helically wound filament portion along the length of the endotracheal tube, and the pitch of the first helically wound filament portion is greater than the pitch of the second helically wound filament portion.

6. The endotracheal tube according to claim 5 wherein the first and second helically wound filament portions are provided as two distinct helically wound filaments sequentially arranged along the length of the endotracheal tube body.

7. The endotracheal tube according to claim 5 wherein one of the first and second helically wound filament portions has a substantially circular cross-sectional shape, and wherein the other of the first and second helically wound filament portions has a flattened cross-sectional shape.

8. The endotracheal tube according to claim 1 wherein the helically wound, meshed or braided reinforcement structure extends along at least 80% of the length of the endotracheal tube.

9. The endotracheal tube according to claim 1 wherein the helically wound, meshed or braided reinforcement structure extends along the ET tube body but does not extend within a distal tip portion of the tube.

10. The endotracheal tube according to claim 1 wherein the filaments comprise a metallic, polymeric, aramid, para-aramid or carbon-fibre material.

11. The endotracheal tube according to claim 1 wherein the endotracheal tube further comprises one or more internal projections, said internal projections projecting radially inwardly from an internal wall surface of a distal tip portion of the endotracheal tube.

12. The endotracheal tube according to claim 1 wherein the endotracheal tube is configured to bend at an applied torque of 1 Nm or less, optionally at an applied torque of 0.75 Nm or less to reach an articulation angle of 50° .

13. The endotracheal tube according to claim 1 wherein the endotracheal tube is configured to resist collapse at an applied pressures up to and including 300 cmH2O.

14. The endotracheal tube according to claim 1 wherein the endotracheal tube further comprises an inflatable cuff provided at or near a distal end of the endotracheal tube.

15. The endotracheal tube according to claim 1 wherein the endotracheal tube body comprises a main body portion and a distal tip portion, said portions being provided as separate components, optionally wherein the main body portion of the ET tube is made from a first material selected from a PVC, thermoplastic elastomer, or silicone material, and the distal tip portion is made from a second material selected from a PVC, thermoplastic elastomer, or silicone, that is different to the first type of material.

16. The endotracheal tube according to claim 1 wherein the endotracheal tube is configured such that the main tube body buckles at a lower applied axial load than the distal tip portion of the device.

17. The endotracheal tube according to claim 1 wherein the endotracheal tube comprises a material having a Shore A Hardness in a range of from 60 to 75.

18. An intubation system comprising an ET tube according to claim 1, and a stylet for guiding an endotracheal tube during intubation.

19. The intubation system of claim 18 wherein the stylet comprises a body having a pivotable tip located at a distal end of the body, the pivotable tip moveable about a pivot point in two opposing directions from the longitudinal axis of the distal end of the stylet body, and a control mechanism for controlling the pivot angle of the pivotable tip.

20. The intubation system according to claim 18 wherein each of the endotracheal tube and the stylet comprise a connector configured for respective engagement with one another to attach the stylet to the endotracheal tube.
